(19) United States
(12) Patent Application Publication
(10) Pub. No.: US 2025/0371256 A1
(43) Pub. Date: Dec. 4, 2025

(54) AUTOMATED FORM FILLING WITH RETRIEVAL AUGMENTED GENERATION FOR MEDICAL DECISION MAKING

(71) Applicant: NEC Laboratories America, Inc., Princeton, NJ (US)

(72) Inventors: Deep Patel, Mount Holly, NJ (US); Erik Kruus, Hillsborough, NJ (US); Iain Melvin, Princeton, NJ (US)

(21) Appl. No.: 19/219,343

(22) Filed: May 27, 2025

Related U.S. Application Data
(60) Provisional application No. 63/652,338, filed on May 28, 2024.

(51) Int. Cl. G06F 40/174; G06F 40/117; G06V 10/75; G06V 10/82; G06V 30/19; G06V 30/412; G16H 10/60

(52) U.S. Cl. CPC G06F 40/174; G06F 40/117; G06V 10/751; G06V 30/19013; G06V 30/412; G16H 10/60; G06V 10/82

(57) ABSTRACT

Methods and systems for filling data include extracting text from a structured document and document instructions to identify a field within the structured document. Text is extracted from a contextual document to identify information relating to the field. Information is selected from the contextual document based on a comparison between the extracted text from the contextual document and the extracted text from the structured document and document instructions. The field within the structured document is filled using the selected information to create a filled document.

### RELATED APPLICATION INFORMATION

This application claims priority to U.S. Patent Application No. 63/652,338, filed on May 28, 2024, incorporated herein by reference in its entirety.

### BACKGROUND

### Technical Field

The present invention relates to large language models and, more particularly, to retrieval augmented generation.

### Description of the Related Art

Structured data can include forms that can be filled with information of a designated type. Completing such structured data can be a time-consuming manual process. Existing language models have difficulty performing this task effectively, as there may be contextual information that needs a high level of comprehension and background information to implement correctly. For example, the different elements of the structured data may have relationships that are not readily apparent from the document itself.

### SUMMARY

A method for filling data includes filling data includes extracting text from a structured document and document instructions to identify a field within the structured document. Text is extracted from a contextual document to identify information relating to the field. Information is selected from the contextual document based on a comparison between the extracted text from the contextual document and the extracted text from the structured document and document instructions. The field within the structured document is filled using the selected information to create a filled document.

A system for filling data includes a hardware processor and a memory that stores a computer program. When executed by the hardware processor, the memory causes the hardware processor to extract text from a structured document and document instructions to identify a field within the structured document, to extract text from a contextual document to identify information relating to the fields, to select information from the contextual document based on a comparison between the extracted text from the contextual document and the extracted text from the structured document and document instructions, and to fill the field within the structured document using the selected information to create a filled document.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of a method for extracting data relating to a structured document, in accordance with an embodiment of the present invention;

FIG. 2 is a block/flow diagram of a method for filling in a structured document, in accordance with an embodiment of the present invention;

FIG. 3 is a block diagram of a healthcare facility where structured document filling can be used to fill forms with patient information, in accordance with an embodiment of the present invention;

FIG. 4 is a block diagram of a computing device that can be used to fill in a structured document, in accordance with an embodiment of the present invention;

FIG. 5 is a diagram of an exemplary neural network structure that may be used to implement part of a large language model, in accordance with an embodiment of the present invention; and

FIG. 6 is a diagram of an exemplary deep neural network structure that may be used to implement part of a large language model, in accordance with an embodiment of the present invention.

### DETAILED DESCRIPTION OF PREFERRED EMBODIMENTS

The information needed to complete structured data can come from multiple sources outside of the document itself, such as a person's personal information (e.g., passport documents, birth certificates, resumes, and medical records). Manually managing and extracting information from such sources can be time-consuming and error-prone. Structured data may instead be completed by an automated system that uses retrieval augmented generation to access these contextual documents and use the information therein, in combination with information gleaned from the structured data document itself, to guide a large language model (LLM) in generating information to complete the structured data. The system may use, e.g., optical character recognition (OCR) to extract information from the contextual documents.

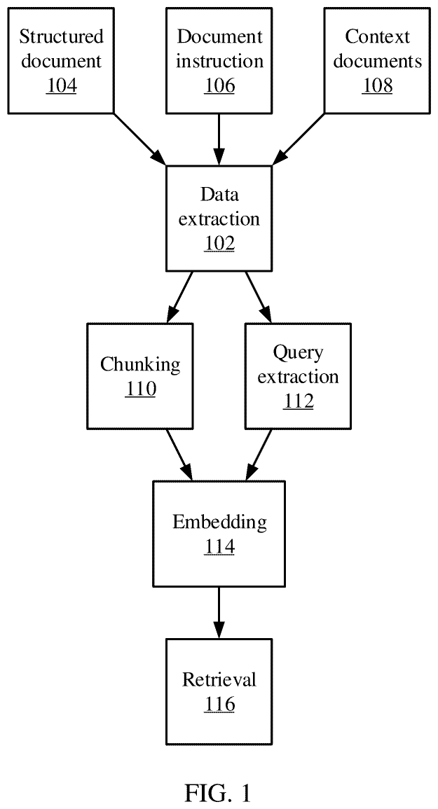

Referring now to FIG. 1, a diagram of extracting information from different sources is shown. Data extraction 102 takes in a structured document 104, instructions 106 relating to the use of the structured document 104, and contextual documents. Data extraction 102 may employ OCR to read out text that is stored in a graphical format and other tools can be used to extract text that is stored in a machine readable format, such as a portable document format (PDF) file. As used herein, the term “structured document” may include textual and/or graphical information as well as one or more fields. The fields may receive and stored information at particular locations of the structured document. The structured document 104 may include information relating to the fields, for example in the form of written instructions to a user or in the form of machine readable information such as a field name.

The data extraction 102 may extract text from contextual documents 108 and the document instructions 106 to identify the text of the instructions 106 and text from relevant references. PDF extraction can extract text and field information from the structured data of the structured document 104, along with corresponding field identifiers.

The OCR of input texts can be performed using any appropriate OCR system, such as a pretrained deep learning model. The PDF information extraction can be implemented by, e.g., converting a PDF document into an extensible markup language (XML) format and using a regular expression-based or conditional parser to extract relevant textual information by its specific location within the structured document 104. The location or lines of text can be identified with a unique identifier for each field and query in the target form.

An output of the data extraction 102, relating in particular to the contextual documents 108, may be chunked into blocks of text with corresponding identifiers. Chunking 110 can be performed using regular expressions or by a natural language processing model that finds and clusters similar text to find chunks with unique information. The regular expressions may be customized based on the field. For example, to extract a name from a person document, a regular expression such as “Name[:\s]+([A-Z][a-z′-]+(?:\s+[A-Z][a-z′-]+){1,3})” may be used. To extract a birth date or other date, a regular such as “\b(\d{1,2}[-/]\d{1,2}[-/](?:19|20)\d{2})\b|\b((?:19|20)\d{2}[- /]\d{1,2}[-/]\d{1,2})\b” may be used. In some cases, an LLM may be sued to extract information from the documents and the regular expressions may be used to verify the LLM output to prevent hallucinations.

Reference text chunks may be embedded in a latent space, and these reference text embeddings may be stored in an embedding database. Embedding 114 may be performed using, e.g., a transformer-based language model that is trained to generate embedding vectors for input text. These vectors are generated so that contextually similar blocks of text are closer together in the latent space. The embedding may be implemented using a deep learning model that is pretrained on diverse text data for next token prediction and masked token prediction. This kind of training helps the model understand the words and sentences while encoding the relationship and meaning of words. Such a transformer-based can output the embedding of words as vectors based on their meaning and context in the text corpus.

Query extraction 112 extracts queries from the target form text with the field identifiers. The extracted target form queries are embedded 114 to output target form query embeddings with field identifiers. The queries include the field identifiers from the original document 104 to assist in filling in relevant information.

Retrieval 116 takes the target form query embeddings as input and retrieves reference information text relevant to answering the query from the embedding database. Retrieval 116 may use, for example, cosine similarity or any other appropriate metric between target query embeddings and the reference information text embeddings in the embedding database to find the most relevant text chunks to the target form query. The similarity measure can be compared to a threshold that determines whether text chunks are sufficiently relevant to the query. Retrieval 116 may be implemented using a natural language processing model that is pretrained for retrieval tasks. The output of this process thus includes text from the instructions 106, text from field identifiers extracted from the structured document 104, and query-relevant text chunks with identifiers.

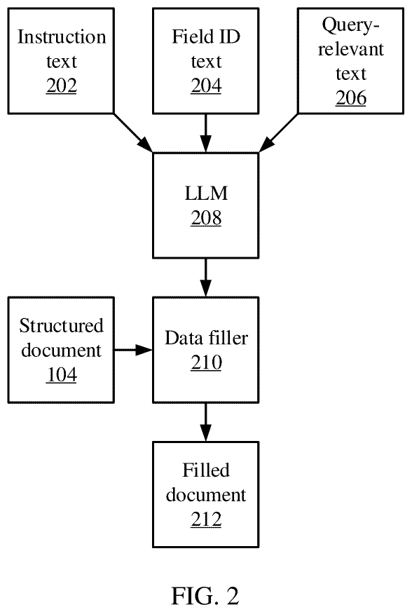

Referring now to FIG. 2, filling the structured document 104 is shown. The instruction text 202, the field identifier text 204, and the query-relevant text chunks 206 are provided in a prompt to an LLM 208. In some embodiments the LLM 208 may be implemented using a deep learning transformer model that is pretrained to predict the next token as well as masked tokens from a large corpus of data from a variety of domains.

The prompt instructs the LLM 208 to answer target form queries using information from the query-relevant text chunks 206 and to follow the instructions from the instruction text 202. Answered queries from the LLM are used by a data filler 210 to fill the empty fields of the structured document 104 using the field identifiers and answers output by the LLM 208. This produces filled document 212, where the fields have been filled with appropriate information drawn from the contextual documents 108. The data filler 210 ensures that the answers from the LLM 208 are of an appropriate data type and may use any appropriate rules and conditions to convert an output to such a data type. For example, if the structured document indicates that a particular field is a check-box (e.g., a representation of binary data), the LLM 208 may answer with a textual answer instead. The data filler 210 converts the text to the appropriate data type for the check-box using the field identifier. This process is repeated for all the fields identifiers of the structured document 104 until the filled document 212 is complete.

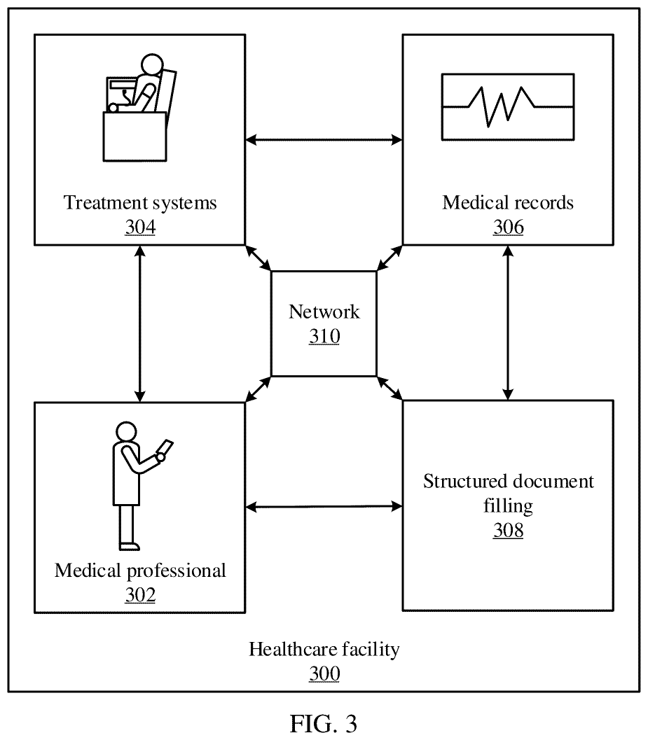

Referring now to FIG. 3, a diagram of therapy generation is shown in the context of a healthcare facility 300. Structured document filling 308 may be used to rapidly collect information from the patient and their medical records 306 for use by medical professionals 302. For example, structured document filling 308 may automatically complete patient intake forms, insurance forms, and emergency contact information. It can also be used for prescription forms to help specialists save time.

The healthcare facility may include one or more medical professionals 302 who review information extracted from a patient's medical records 306 to determine their healthcare and treatment needs. These medical records 306 may include self-reported information from the patient, test results, and notes by healthcare personnel made to the patient's file. Treatment systems 304 may furthermore monitor patient status to generate medical records 306 and may be designed to automatically administer and adjust treatments as needed.

Medical professionals 302 may use the structured document filling 308 to provide customized healthcare that is tailored to the patient's needs. For example, the medical professionals 302 may use structured document filling 308 to collect information about a patient in one place, so that the medical professional can access an accurate summary of the patient's condition and medical history.

The different elements of the healthcare facility 300 may communicate with one another via a network 310, for example using any appropriate wired or wireless communications protocol and medium. Thus the structured document filling 308 can be used to collect information from disparate sources, using test results and medical records 306. The treatment systems 304 may be used to generate and administer a therapy based on the filled documents generated by structured document filling 308.

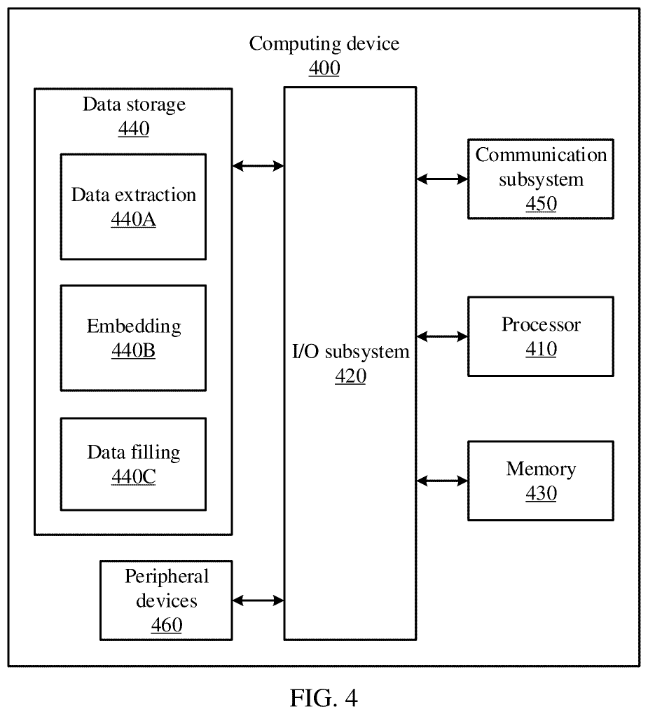

As shown in FIG. 4, the computing device 400 illustratively includes the processor 410, an input/output subsystem 420, a memory 430, a data storage device 440, and a communication subsystem 450, and/or other components and devices commonly found in a server or similar computing device. The computing device 400 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 430, or portions thereof, may be incorporated in the processor 410 in some embodiments.

The processor 410 may be embodied as any type of processor capable of performing the functions described herein. The processor 410 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 430 may be embodied as any type of volatile or non-volatile memory or data storage capable of performing the functions described herein. In operation, the memory 430 may store various data and software used during operation of the computing device 400, such as operating systems, applications, programs, libraries, and drivers. The memory 430 is communicatively coupled to the processor 410 via the I/O subsystem 420, which may be embodied as circuitry and/or components to facilitate input/output operations with the processor 410, the memory 430, and other components of the computing device 400. For example, the I/O subsystem 420 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 420 may form a portion of a system-on-a-chip (SOC) and be incorporated, along with the processor 410, the memory 430, and other components of the computing device 400, on a single integrated circuit chip.

The data storage device 440 may be embodied as any type of device or devices configured for short-term or long-term storage of data such as, for example, memory devices and circuits, memory cards, hard disk drives, solid state drives, or other data storage devices. The data storage device 440 can store program code 440A for data extraction, 440B for performing embedding, and/or 440C for filling data in structured documents. Any or all of these program code blocks may be included in a given computing system. The communication subsystem 450 of the computing device 400 may be embodied as any network interface controller or other communication circuit, device, or collection thereof, capable of enabling communications between the computing device 400 and other remote devices over a network. The communication subsystem 450 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 400 may also include one or more peripheral devices 460. The peripheral devices 460 may include any number of additional input/output devices, interface devices, and/or other peripheral devices. For example, in some embodiments, the peripheral devices 460 may include a display, touch screen, graphics circuitry, keyboard, mouse, speaker system, microphone, network interface, and/or other input/output devices, interface devices, and/or peripheral devices.

Of course, the computing device 400 may also include other elements (not shown), as readily contemplated by one of skill in the art, as well as omit certain elements. For example, various other sensors, input devices, and/or output devices can be included in computing device 400, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 400 are readily contemplated by one of ordinary skill in the art given the teachings of the present invention provided herein.

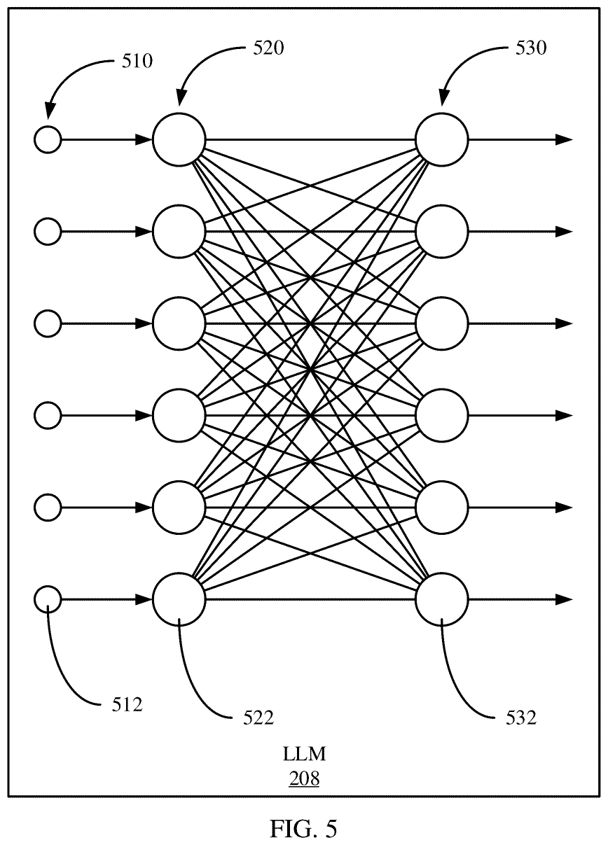

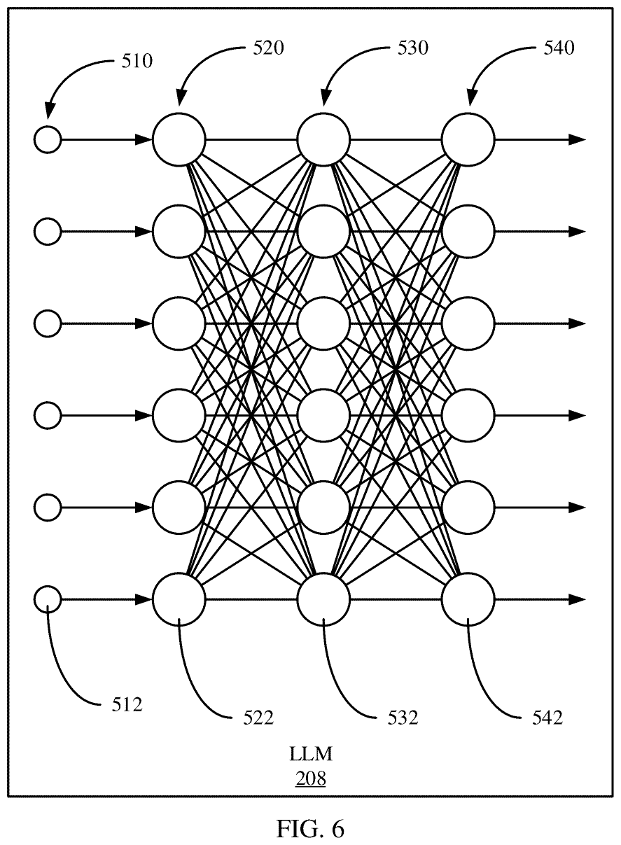

Referring now to FIGS. 5 and 6, exemplary neural network architectures are shown, which may be used to implement parts of the present models, such as the LLM 208. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 520 of source nodes 522, and a single computation layer 530 having one or more computation nodes 532 that also act as output nodes, where there is a single computation node 532 for each possible category into which the input example could be classified. An input layer 520 can have a number of source nodes 522 equal to the number of data values 512 in the input data 510. The data values 512 in the input data 510 can be represented as a column vector. Each computation node 532 in the computation layer 530 generates a linear combination of weighted values from the input data 510 fed into input nodes 520, and applies a non-linear activation function that is differentiable to the sum. The exemplary simple neural network can perform classification on linearly separable examples (e.g., patterns).

A deep neural network, such as a multilayer perceptron, can have an input layer 520 of source nodes 522, one or more computation layer(s) 530 having one or more computation nodes 532, and an output layer 540, where there is a single output node 542 for each possible category into which the input example could be classified. An input layer 520 can have a number of source nodes 522 equal to the number of data values 512 in the input data 510. The computation nodes 532 in the computation layer(s) 530 can also be referred to as hidden layers, because they are between the source nodes 522 and output node(s) 542 and are not directly observed. Each node 532, 542 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for filling data, comprising:
extracting text from a structured document and document instructions to identify a field within the structured document;
extracting text from a contextual document to identify information relating to the field;
selecting information from the contextual document based on a comparison between the extracted text from the contextual document and the extracted text from the structured document and document instructions; and
filling the field within the structured document using the selected information to create a filled document.

2. The method of claim 1, wherein extracting text from the contextual document includes chunking text from the contextual document into blocks using a regular expression.

3. The method of claim 2, wherein extracting text from the contextual document uses a large language model to identify relevant information and uses the regular expression to validate an output of the large language model.

4. The method of claim 3, wherein the large language model is implemented as a machine learning model.

5. The method of claim 1, wherein filling the field includes prompting a large language model using the text from the structured document and the document instructions as well as the text from the contextual document to answer form queries using information from the contextual document in accordance with the document instructions.

6. The method of claim 1, wherein extracting text from the structured document and document instructions includes using optical character recognition (OCR) to identify text that is stored in a graphical format.

7. The method of claim 1, wherein extracting text from the structured document and document instructions includes extracting machine-readable text from a file.

8. The method of claim 1, wherein selecting information includes comparing an embedding of a query based on the text from the structured document and document instructions with an embedding of the text from the contextual document.

9. The method of claim 1, wherein the contextual document is a personal record of a patient that includes identifying information.

10. The method of claim 9, wherein the structured document is a healthcare form used for medical decision making.

11. A system for filling data, comprising:
a hardware processor; and
a memory that stores a computer program which, when executed by the hardware processor, causes the hardware processor to:
extract text from a structured document and document instructions to identify a field within the structured document;
extract text from a contextual document to identify information relating to the field;
select information from the contextual document based on a comparison between the extracted text from the contextual document and the extracted text from the structured document and document instructions; and
fill the field within the structured document using the selected information to create a filled document.

12. The system of claim 11, wherein extraction of text from the contextual document includes chunking text from the contextual document into blocks using a regular expression.

13. The system of claim 12, wherein extraction of text from the contextual document uses a large language model to identify relevant information and uses the regular expression to validate an output of the large language model.

14. The system of claim 13, wherein the large language model is implemented as a machine learning model.

15. The system of claim 11, wherein the filling of the field includes a prompt to a large language model using the text from the structured document and the document instructions as well as the text from the contextual document to answer form queries using information from the contextual document in accordance with the document instructions.

16. The system of claim 11, wherein extraction of text from the structured document and document instructions includes using optical character recognition (OCR) to identify text that is stored in a graphical format.

17. The system of claim 11, wherein extraction of text from the structured document and document instructions includes extracting machine-readable text from a file.

18. The system of claim 11, wherein selection of information includes comparing an embedding of a query based on the text from the structured document and document instructions with an embedding of the text from the contextual document.

19. The system of claim 11, wherein the contextual document is a personal record of a patient that includes identifying information.

20. The system of claim 19. wherein the structured document is a healthcare form used for medical decision making.
